(19) United States
(12) Patent Application Publication
(10) Pub. No.: US 2026/0138954 A1
(43) Pub. Date: May 21, 2026

(54) AMORPHOUS SOLID FORM AND PHARMACEUTICAL COMPOSITIONS OF LENVATINIB BESYLATE

(71) Applicant: Synthon B.V., Nijmegen (NL)

(72) Inventors: Carles SOLE, Sant Boi de Llobregat (ES); Lisardo ALVAREZ FERNANDEZ, Sant Boi de Llobregat (ES); Rohit KUMAR, Sant Boi de Llobregat (ES); Michal HEGEDUS, Blansko (CZ)

(21) Appl. No.: 19/394,501

(22) Filed: Nov. 19, 2025

(30) Foreign Application Priority Data
Aug. 18, 2025 (EP) 25196466.4

(51) Int. Cl. C07D 215/48; A61K 9/16; A61K 9/48; A61K 31/47

(52) U.S. Cl. CPC C07D 215/48; A61K 9/1623; A61K 9/1652; A61K 9/1694; A61K 9/4858; A61K 9/4866; A61K 31/47

(57) ABSTRACT

The invention relates to amorphous solid forms and pharmaceutical compositions comprising amorphous lenvatinib besylate (benzene sulfonate), and the processes for their preparation.

The invention relates to solid forms of lenvatinib besylate (benzenesulfonate), specifically to amorphous lenvatinib besylate and to pharmaceutical compositions comprising it. It further relates to processes for preparation of the amorphous form and solid oral dosage forms that exhibit superior stability, dissolution, and bioavailability while avoiding gelation.

### BACKGROUND OF THE PRESENT INVENTION

Lenvatinib, 4-[3-chloro-4-(cyclopropylcarbamoylamino)phenoxy]-7-methoxyquinoline-6-carboxamide of Formula (I),

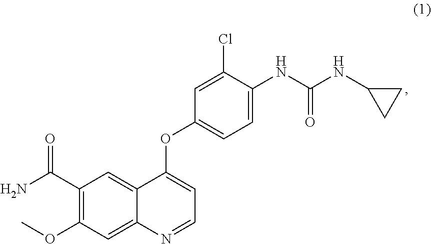 [SMILES: COc1cc2nccc(Oc3ccc(NC(=O)NC4CC4)c(Cl)c3)c2cc1C(N)=O]

is a receptor tyrosine kinase (RTK) inhibitor that selectively inhibits the kinase activities of vascular endothelial growth factor (VEGF) receptors VEG FRI, VEGFR2 and VEGFR3.

The mesylate salt of lenvatinib (lenvatinib mesylate) is marketed as Lenvima®/Kisplyx® and is approved for thyroid carcinoma, renal cell carcinoma, hepatocellular carcinoma and endometrial carcinoma.

Lenvatinib was first disclosed in WO2002/032872 by Eisai. In WO2005/063713 several lenvatinib salts were disclosed including hydrochloride, hydrobromide, tosylate, sulphate, esylate and mesylate. Crystalline Forms A, Band C of lenvatinib mesylate and several mesylate (hydrated Form F, DMSO solvate, HAC-solvate Form I) and esylate solvates were described. Amorphous forms of lenvatinib methane sulfonate and ethane sulfonate are described in WO2006/137474.

However, lenvatinib mesylate is polymorphic, hygroscopic, and prone to gelation in aqueous media. Upon contact with water, crystalline particles dissolve rapidly at the surface, causing local supersaturation and molecular self-assembly through hydrogen bonding and π-π stacking into a supramolecular fibrous network (hydrogel), delaying release. This phenomenon makes processing and consistent drug release challenging.

WO 2020/070144 A1 discloses crystalline lenvatinib besylate Forms 1 (anhydrous) and 2 (DMSO solvate). Form 1 is prone to electrostatic charging, hindering blending and capsule filling. Form 2, while well-crystallized, may undergo desolvation under manufacturing stress, transforming to Form 1 and exhibiting solubility shifts under humidity and heat. These transformations limit reproducibility and stability.

Accordingly, there remains a need for a stable solid form of lenvatinib besylate with improved processability and dissolution, resistant to polymorph conversion and gelation.

There remains a need for a solid form of lenvatinib besylate that is both stable and readily processable, and that provides consistent dissolution and bioequivalent exposure without the gelation seen in crystalline salts.

### BRIEF DESCRIPTION OF THE INVENTION

The present invention provides an amorphous solid form of lenvatinib besylate, characterized by an XRPD pattern lacking sharp diffraction peaks.

The amorphous form may be obtained by any process, including but not limited to melt-quenching or hot-melt extrusion, solvent evaporation, spray drying, co-precipitation, mechanical milling, or in-process transformation during granulation.

The invention further provides solid oral dosage forms, particularly capsules, comprising amorphous lenvatinib besylate and pharmaceutically acceptable excipients such as alkalizers, diluents, disintegrants, and lubricants. These compositions are stable, show no gelation in aqueous dissolution, and are bioequivalent to Lenvima® capsules.

### BRIEF DESCRIPTION OF THE DRAWINGS

FIG. 1 depicts the X-Ray Powder Diffractogram (XRPD) of solid Form III of Lenvatinib besylate.

FIG. 2 depicts the DSC pattern of solid Form III of Lenvatinib besylate.

### DETAILED DESCRIPTION OF THE INVENTION

AMORPHOUS lenvatinib besylate is a non-crystalline, single-phase solid. It may be prepared from any crystalline form (e.g., Form 1, 2, III or mixtures thereof) using thermal, solvent, or mechanical methods. XRPD exhibits a featureless pattern; The NEW solid form III can be characterized by XRPD pattern having 2θ values 4.7°, 14.2° and 16.5° degrees 2 theta (±0.2 degrees 2 theta). The solid Form III can be also characterized by XRPD pattern having 2θ values 4.7°, 14.2°, 15.1°, 16.5°, 23.0° and 26.3° degrees 2 theta (±0.2 degrees 2 theta). The solid form can be further characterized by XRPD pattern described in the following table:

| Angle | Intensity |
|---|---|
| (2-Theta °) | (%) |
| 4.7 | 100.0 |
| 5.1 | 2.6 |
| 9.5 | 11.6 |
| 10.3 | 7.1 |
| 11.7 | 1.6 |
| 12.8 | 7.5 |
| 13.4 | 5.9 |
| 13.7 | 3.4 |
| 14.2 | 49.8 |
| 14.8 | 9.5 |
| 15.1 | 23.6 |
| 16.2 | 8.1 |
| 16.5 | 30.0 |
| 16.9 | 14.0 |
| 17.2 | 7.3 |
| 17.8 | 1.9 |
| 18.8 | 3.5 |
| 19.0 | 2.6 |
| 19.9 | 7.0 |
| 20.3 | 5.7 |
| 20.6 | 8.5 |
| 21.2 | 37.3 |
| 21.4 | 17.7 |
| 22.0 | 9.0 |
| 22.4 | 9.0 |
| 23.0 | 21.3 |
| 24.0 | 12.5 |
| 24.4 | 14.2 |
| 24.9 | 8.2 |
| 25.3 | 4.9 |
| 25.7 | 5.7 |
| 26.3 | 29.6 |
| 27.2 | 13.8 |
| 27.6 | 6.9 |
| 28.4 | 5.1 |
| 28.9 | 9.1 |
| 29.4 | 3.4 |
| 29.9 | 2.2 |
| 30.7 | 7.4 |
| 31.2 | 3.5 |
| 31.5 | 2.4 |
| 32.0 | 1.9 |
| 32.5 | 2.3 |
| 33.1 | 2.3 |
| 33.6 | 2.6 |
| 34.2 | 4.2 |

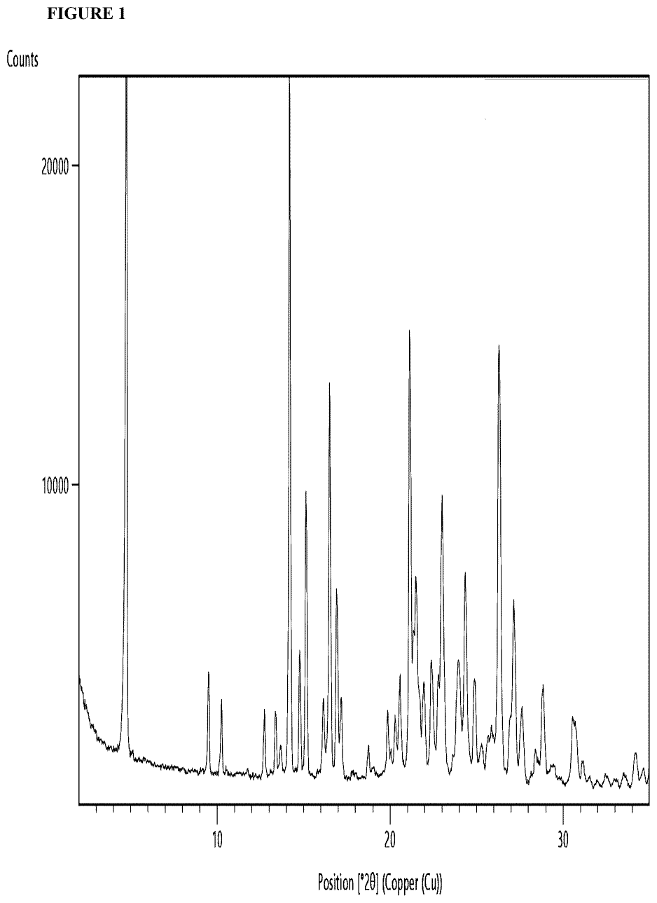

The solid Form III can be also characterized by XR-PD pattern depicted in FIG. 1.

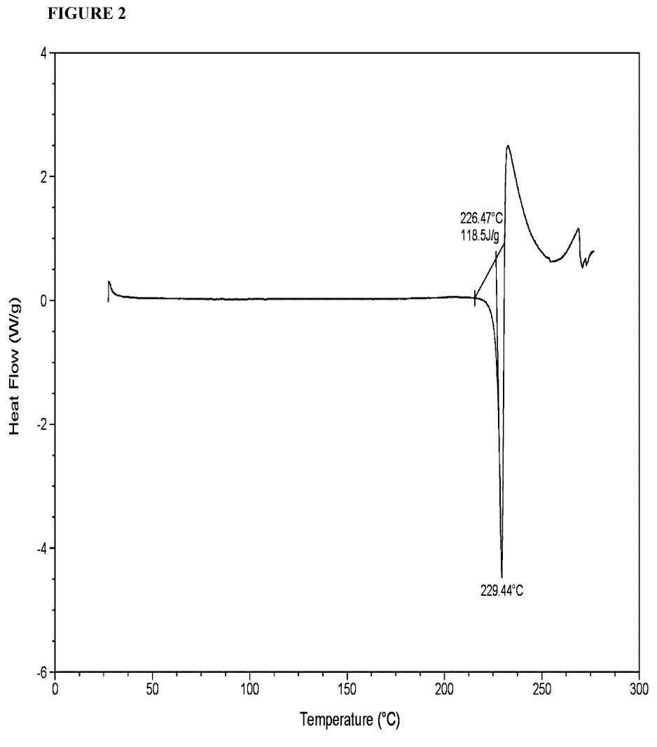

The solid Form III can be further characterized by DSC pattern depicted in FIG. 2.

The solid Form III can be prepared by a process comprising storing a crystalline form of lenvatinib besylate (for example Form I described in WO2020/070144) in the humidity chamber in low density polyethylene (LDPE) bag at temperature 55° C. and humidity 90% relative humidity for 12 days.

Form III of lenvatinib besylate has similar solubility as the Form 1 described in WO2020/070144. Therefore, mixtures of both forms can be used for instance in the preparation of a lenvatinib besylate amorphous or in the preparation of pharmaceutical formulations.

The amorphous form of the invention displays higher apparent solubility than crystalline forms, no gelation during dissolution testing, and chemical and physical stability under accelerated storage (40° C./75% RH, 6 months).

Lenvatinib besylate's large, aromatic benzenesulfonate anion increases the glass-transition temperature and suppresses recrystallization. Amorphisation removes long-range order while preserving ionic pairing, giving a kinetically stable disordered salt. The form remains amorphous under typical processing and storage stresses.

The amorphous lenvatinib besylate of the present invention offers multiple advantages over existing crystalline besylate and mesylate forms. Unlike lenvatinib mesylate, which tends to form viscous gels upon contact with aqueous media, the amorphous besylate disperses rapidly and shows no gelation, ensuring uniform and predictable dissolution. It demonstrates excellent physical stability, remaining fully amorphous after six months of storage at 40° C. and 75% relative humidity, with no detectable recrystallization or new XRPD peaks. In bioequivalence studies, the capsule formulation exhibited bioequivalence to the marketed Lenvima® capsules. Furthermore, the amorphous besylate displays superior manufacturability, handling well during blending and encapsulation, avoiding electrostatic charging, and eliminating the need for polymeric carriers typically required to stabilize amorphous systems. The amorphous form may be obtained by any process, including but not limited to melt-quenching or hot-melt extrusion, solvent evaporation, spray drying, co-precipitation, mechanical milling/grinding, or in-process transformation during granulation. For instance the lenvatinib besylate amorphous can be prepared with a process comprising:
1. Heating lenvatinib besylate to a temperature 230° C. at a rate between 5° C./min and 20° C./min;
2. Cooling the mixture either:
   a. To a temperature between 20° C. and 25° C. at a rate between 10° C./min and 25° C./min; or
   b. By contacting the mixture with liquid nitrogen.

Heating of lenvatinib besylate to a temperature 230° C. can be done at a rate between 5° C./min and 20° C./min, preferably between 10° C./min and 20° C./min. Obtained mixture can be optionally hold at temperature 230° C. for between 0.5 min and 3 minutes. The mixture is then cooled either
a. To a temperature between 20° C. and 25° C. C at a rate between 10° C./min and 25° C./min; or
b. By contacting the mixture with liquid nitrogen;
to obtain amorphous solid of lenvatinib besylate.

In another embodiment crystalline lenvatinib besylate may be milled by grinding action between two surfaces till obtain amorphous solid. In pharmaceutical industry generally done by ball milling, jet milling, or other high-energy milling techniques.

These methods reproducibly supply a lattice disrupting energy at scale, enabling amorphisation under controlled, GMP compliant conditions.

The amorphous solid lenvatinib besylate can be processed into a suitable pharmaceutical composition.

A further aspect of the present invention relates to a pharmaceutical composition comprising a therapeutically effective dose of amorphous lenvatinib besylate that is stable and are bioequivalent to the commercial lenvatinib mesylate capsules (Lenvima®).

The pharmaceutical composition comprising the therapeutically effective dose of amorphous Lenvatinib besylate may further comprise sodium carbonates in a weight ratio of amorphous lenvatinib besylate to sodium carbonates ranges from 1:1.5 to 1:10, more preferably from 1:2 to 1:7 most preferred ranges are from 1:3 to 1:5.

Besides sodium carbonates one or more pharmaceutically acceptable excipients can be used additionally in accordance with the present invention.

In a preferred embodiment sodium carbonates are used in an amount of 2% to 65%, preferably 5% to 55%, more preferably 20% to 50%, most preferably 25% to 50% by weight based on the total weight of the composition.

The one or more pharmaceutically acceptable excipients to be used additionally to sodium carbonates in accordance with the present invention can be chosen from, for example, diluents, binders, disintegrants, lubricants, and glidants.

In a preferred embodiment, amorphous lenvatinib besylate pharmaceutical composition can be mixed with pharmaceutically acceptable adjuvants, diluents or carriers.

Diluents are fillers which are used to increase the bulk volume of a tablet or capsule. By combining a diluent with the active pharmaceutical ingredient, the final product is given adequate weight and size to assist in production and handling. Binders hold the excipients that are present in a tablet/granule together.

The pharmaceutical composition of the present invention preferably contains at least one diluent.

Diluents are preferably used in an amount of from 15% to 75%, preferably 20% to 50%, more preferably 22% to 45%, by weight based on the total weight of the composition. Suitable examples of diluents to be used in accordance with the present invention include starch, pregelatinized starch, microcrystalline cellulose (MCC), mannitol, and calcium phosphate.

In a preferred embodiment of the present invention, the diluents to be used are mannitol, microcrystalline cellulose or mixtures thereof.

The pharmaceutical composition of the present invention may also contain a binder. Binders ensure that tablets and granules can be formed having the desired or required mechanical strength. Binders which are suitable for use in accordance with the present invention include povidone, hydroxypropyl cellulose, hydroxypropyl methylcellulose, and sodium carboxyl methylcellulose. Binders are preferably used in an amount of from 1% to 6% by weight based on the total weight of the composition.

The pharmaceutical composition of the present invention may also contain a disintegrant. Disintegrants are added to a tablet or capsule composition to promote the breakup of the tablet/capsule into smaller fragments in an aqueous environment, thereby increasing the available surface area and promoting a more rapid release of the active pharmaceutical ingredient. Suitable examples of disintegrants to be used in accordance with the present invention include crospovidone, L-HPC (Low substituted hydroxypropyl cellulose), sodium starch glycolate, croscarmellose sodium, and mixtures of any of the foregoing. Disintegrants preferably are used in an amount of from 5% to 40% by weight based on the total weight of the composition; the amount will depend on the tablet size and the chosen disintegrant. A preferred disintegrant is low substituted hydroxypropyl cellulose, in a preferred amount of from 15% to 35% by weight based on the total weight of the composition.

The pharmaceutical composition of the invention may also contain a lubricant. Lubricants are generally used in order to reduce sliding friction. In particular, to decrease the friction at the interface between the blend to be encapsulated and dosator of the encapsulation machine. Suitable lubricants to be used in accordance with the present invention include magnesium stearate, stearic acid, glyceryl behenate, hydrogenated vegetable oil, talc and glycerine fumarate. A preferred lubricant is talc. The pharmaceutical composition of the invention may also contain a glidant. Glidants enhance product flow by reducing interparticulate friction. A suitable example is colloidal silicon dioxide.

Lubricants and glidants preferably are used in a total amount of from 0.05% to 5% by weight based on the total weight of the composition, preferably from 1% to 5%.

In a preferred embodiment of the present invention, the amount of amorphous lenvatinib besylate in the composition depends on the condition and the patient to be treated. Typically, the pharmaceutical composition comprises an amount of amorphous lenvatinib besylate equivalent to 4 mg to 24 mg of lenvatinib free base per unit dose, administered once daily, with the exact dosage adjusted according to the type of malignancy, disease progression, and the patient's clinical response. For example oral dosage forms, with dose strength of 4 mg, 8 mg, 10 mg, 12 mg, 14 mg, 18 mg, 20 mg and 24 mg may be used.

The pharmaceutical composition can be in the form of a solid oral composition, for example a capsule, a tablet, a powder or a granule. The composition in a form of a tablet or a capsule or a granule can be coated. In a preferred embodiment the solid oral composition is a capsule.

The compositions of the present invention can be prepared by direct mixing or granulating the Lenvatinib besylate with one or more pharmaceutically acceptable excipients, optionally followed by encapsulation, using equipment and methods well-known to the skilled artisan.

In a preferred embodiment the composition is prepared by granulation process.

Granulation can be performed by a wet or dry process. The wet granulation uses water or organic solvents or mixtures thereof as granulation liquid. The dry granulation can be performed by processes known as slugging and/or roller compaction.

The pharmaceutically acceptable excipients to be used in accordance with the present invention, can be used only intragranularly, only extragranularly, or both.

The same formulations and process described above are applicable when lenvatinib is used in its crystalline form, which may be converted into the amorphous state during processing.

In an alternative embodiment the starting lenvatinib besylate active ingredient can be crystalline and transformed into amorphous lenvatinib beyslate during the drug product manufacturing.

Any crystalline lenvatinib besylate form can be used as a starting API to form amorphous lenvatinib besylate solid oral composition, preferably capsules.

For instance crystalline Form 1 or Form 2 or Form III of lenvatinib besylate or mixtures thereof can be used as an API.

In a preferred embodiment, granulation is used as a manufacturing process. During manufacturing, particularly in granulation (wet or dry), materials undergo mechanical stress, moisture exposure, and sometimes heat, all of which can disrupt the long-range order of a crystal lattice. The mechanical stress arises from the impact of high-shear mixing, compression, or particle to particle collisions occurring during the granulation process. Such stress, in combination with localized thermal energy and the presence of moisture, can induce a complete transformation of the crystalline lenvatinib besylate into its amorphous form.

This transformation occurs due to disruption of the intermolecular interactions within the crystal lattice, resulting in molecular disorder and formation of an amorphous phase.

When wet granulation is used, during drying, residual moisture and elevated temperature increase molecular mobility. The molecules, freed from lattice constraints, can be trapped in a disordered amorphous state as the solvent evaporates rapidly.

This is particularly pronounced for compounds with low recrystallization tendency or high glass transition temperature (Tg), like some salt forms (e.g., besylate salts).

In a preferred embodiment the pharmaceutical composition is a capsule comprising:

| Component | % w/w |
|---|---|
| Amorphous lenvatinib besylate | 4-25 |
| Sodium hydrogen carbonate | 20-50 |
| Microcrystalline cellulose (MCC) | 10-50 |
| Low-substituted HPC | 15-35 |
| Mannitol | 5-10 |
| Talc | 1-5 |

Granulation with water or ethanol/water mixture, drying, and encapsulation yield homogeneous, free-flowing granules containing amorphous lenvatinib besylate adsorbed on MCC and L-HPC.

The invention will be further described with reference to the following examples.

### Examples

DCS patterns were obtained using the following conditions: 10° C./min->250° C.

XRPD spectrum was obtained using the following measurement conditions: Panalytical Empyrean diffractometer with Θ/2Θ geometry (transmition mode), equipped with a PixCell 3D detector;

| Start angle (2θ): | 2.0° | |
|---|---|---|
| End angle (2θ): | 35.0° | |
| Step size: | 0.026° | |
| Scan speed: | 0.0955°/seconds | |
| Radiation type: | Cu | |
| Radiation wavelengths: | 1.5406 Å (Kα1), primary monochromator used | |
| Divergence slit: | 1/2° | |
| Antiscatter slit: | 1/2° | |
| Soller slit: | 0.02 | rad |
| Detector slit: | 7.5 | mm |
| Rotation speed: | 30 | rpm |

### Example 1: Preparation of Solid Amorphous Form of Lenvatinib Besylate

0.1 g of crystalline lenvatinib besylate was heated in controlled manner (10° C./min) to 230° C. The mixture was holt at 230° C. for 1 minute. The mixture was then cooled down to 25° C. at rate 20° C./min. Amorphous lenvatinib besylate was obtained.

### Example 2: Preparation of Solid Form III of Lenvatinib Besylate

25 g Form 1 of Lenvatinib besylate was stored in the humidity chamber in sealable LDPE bag (height×width: 250×350 mm, thickness of the wall 40 μm) at 55° C. and humidity 90% of relative humidity for 12 days. The material was spread out in the bag so that the height of its layer was as small as possible. XRPD of obtained solid Form III is depicted in FIG. 1, DSC is depicted in FIG. 2.

### Example 3: Pharmaceutical Compositions of Amorphous Lenvatinib Besylate

| | Dose strength | |
|---|---|---|
| | 10 mg | |
| Ingredients | mg/cap | % |

Intragranular phase

| | | |
|---|---|---|
| Lenvatinib besylate | 13.706 | 10.964 |
| Sodium hydrogen carbonate | 41.250 | 33.000 |
| Mannitol | 8.750 | 7.000 |
| Microcrystalline cellulose (MCC) | 11.272 | 9.018 |
| Low-substituted hydroxypropyl cellulose (L-HPC) | 15.625 | 12.500 |
| Hydroxypropyl cellulose (HPC) | 3.750 | 3.000 |

Extragranular phase

| | | |
|---|---|---|
| Microcrystalline cellulose | 11.272 | 9.018 |
| Low-substituted hydroxypropyl cellulose | 15.625 | 12.500 |
| Talc | 3.750 | 3.000 |
| Capsule content weight | 125.000 | 100.000 |

219 grams of lenvatinib besylate mixture of Form 1 (described in WO 2020/070144) and III were mixed with 660 grams of NaHCO₃, 140 gr of mannitol, 180 gr of MCC, 250 gr of L-TIPC and 60 gr of HPC. A wet granulation with water was performed in a high-shear mixer and later dried in a fluidized bed. The resulting granules were milled and re-blended with the extragranular components (180 gr MCC, 250 gr L-HPC and 60 gr of talc). Final blend was encapsulated in a dosator capsule machine. A capsule containing amorphous lenvatinib besylate was obtained.

Results fasting study composition of example 3 containing amorphous lenvatinib besylate

| | Geometric Means T/R* |
|---|---|
| Pharmacokinetic | (90% confidence interval) (%) |
| Parameter | Prototype A′ |
| Cₘₐₓ | 96.09 |
| | (82.15-112.40) |
| AUC₇₂ | 95.55 |
| | (87.63-104.18) |

1. An amorphous solid form of lenvatinib besylate.

2. A pharmaceutical composition comprising a therapeutically effective dose of amorphous lenvatinib besylate according to claim 1 and pharmaceutically acceptable excipients.

3. The pharmaceutical composition according to claim 2 further comprising sodium carbonates having a weight ratio of lenvatinib besylate amorphous to sodium carbonates in a range from 1:1.5 to 1:10.

4. The pharmaceutical composition according to claim 2 wherein the composition further comprises:
a) at least one diluent in an amount of from 15% to 75% based on the total weight of the composition;
b) optionally, disintegrant in an amount of from 1% to 35% by weight based on the total weight of the composition; and
c) lubricant in an amount of from 1% to 5% by weight based on the total weight of the composition.

5. The pharmaceutical composition according to claim 4 wherein the diluent is MCC, mannitol or a mixture of both.

6. The pharmaceutical composition according to claim 2 comprising, based on the total weight of the composition:
a) a therapeutically effective dose of amorphous lenvatinib besylate in an amount of from 4% to 25% by weight;
b) microcrystalline cellulose in an amount of from 10% to 50% by weight;
c) sodium carbonates, in an amount of from 20% to 50% by weight;
d) low substituted hydropropyl cellulose in an amount of from 15% to 35% by weight;
e) mannitol in an amount of from 5% to 10% by weight; and
f) talc in an amount of from 1% to 5% by weight.

7. The pharmaceutical composition according to claim 6 prepared by wet granulation process with aqueous or hydroalcoholic solvent.

8. The composition of claim 6 wherein the lenvatinib besylate is adsorbed onto MCC or L-HPC surfaces.

9. The pharmaceutical composition according to claim 2 which is a capsule.

10. A process for preparing amorphous lenvatinib besylate comprising heating or milling or solvent evaporation or granulation of a crystalline lenvatinib besylate until an amorphous phase is obtained.

11. The process for preparing amorphous lenvatinib besylate according to claim 10, the process comprising the steps of:
a) heating the crystalline lenvatinib besylate to a temperature 230° C. at a rate between 5° C./min and 20° C./min;
b) cooling the mixture either;
to a temperature between 20° C. and 25° C. at a rate between 10° C./min and 25° C./min; or
by contacting the mixture with liquid nitrogen.

12. A solid Form III of lenvatinib besylate characterized by XRPD pattern having 2θ values 4.7°, 14.2° and 16.5° degrees 2 theta (±0.2 degrees 2 theta).

13. The solid Form III according to claim 12 characterized by XRPD pattern having 2θ values 4.7°, 14.2°, 15.2°, 16.5°, 23.0° and 26.3° degrees 2 theta (±0.2 degrees 2 theta).

14. A method of preparing a pharmaceutical composition according to claim 2, which comprises granulating a crystalline form of lenvatinib besylate with at least one excipient to form granules, wherein said granulation is sufficient to transform said crystalline Lenvatinib besylate into amorphous lenvatinib besylate.

15. The method according to claim 14, wherein the crystalline form is Form 1, Form III or a mixture thereof.

16. A method of treating thyroid carcinoma, hepatocellular carcinoma, renal cell carcinoma or endometrial carcinoma, which comprises administering an effective amount of the amorphous lenvatinib besylate of claim 1 to a patient in need thereof.
